(19) United States
(12) Patent Application Publication
(10) Pub. No.: US 2025/0248615 A1
(43) Pub. Date: Aug. 7, 2025

(54) BIO-IMPEDANCE MEASUREMENT SYSTEM

(71) Applicant: Analog Devices, Inc., Wilmington, MA (US)

(72) Inventors: Yaohua Yang, West Linn, OR (US); Ruming Yin, Portland, OR (US); Yi Zhang, Portland, OR (US)

(73) Assignee: Analog Devices, Inc., Wilmington, MA (US)

(21) Appl. No.: 19/034,255

(22) Filed: Jan. 22, 2025

Related U.S. Application Data
(60) Provisional application No. 63/550,542, filed on Feb. 6, 2024.

(51) Int. Cl. A61B 5/053; A61B 5/00; G16H 40/63; G16H 50/30

(52) U.S. Cl. CPC A61B 5/053; A61B 5/7225; A61B 5/7246; G16H 40/63; G16H 50/30

(57) ABSTRACT

A measurement system and method can include: a signal generator configured to provide a transmit voltage; a transmit electrode connected to the signal generator; a trans-impedance amplifier having a trans-impedance amplifier input and a trans-impedance amplifier output; a receive electrode connected to the trans-impedance amplifier input; voltage measurement circuitry configured to: acquire a transmit voltage measurement from a first connection connected between the signal generator and the transmit electrode and from a second connection connected to the trans-impedance amplifier input, and acquire a receive voltage measurement from a third connection connected to the trans-impedance amplifier input and from a fourth connection connected to the trans-impedance amplifier output; and a digital processing unit configured to determine an impedance based on the electrodes being in direct contact with a body, the impedance based on a ratio of the transmit voltage measurement and the receive voltage measurement.

### TECHNICAL FIELD

This disclosure relates to bio-impedance measurements, more particularly to correlated bio-impedance measurements.

### BACKGROUND

Impedance measurements of the body, referred herein as bio-impedance, have many applications in healthcare and consumer goods for providing non-invasive measurements of the heart and blood vessels. Bio-impedance measurements can, for example, provide important health related monitoring of cardiac volumetric output cycle by cycle, vascular resistance to blood flow, and fluid levels in the body.

These measurements are useful for the purposes of detecting signs of pulmonary edema or assessing body composition. Bio-impedance measurements can be made by electrodes provided in wearable devices, such as wrist watches, chest bands, head bands, and patches.

These bio-impedance measurements are part of electrical impedance tomography and are an emerging non-invasive technique of medical imaging, but many challenges remain. These challenges generally result in inaccurate measurements and high power usage at higher frequencies.

One inaccuracy results from AC source output impedance, which contributes to error and can change from measurement to measurement. Another source of inaccuracy results from differing electrode condition when in contact with a body, meaning the electrodes could be dirty, wet, sweaty, or even very dry. Each of these different conditions can introduce inaccuracy into the measurements.

Yet another source of inaccuracy results from the presence of board parasitic capacitance and from the finite bandwidth of trans-impedance amplifiers used within most bio-impedance measurement systems. As such, prior solutions include bandwidth limitations from transmit and receive path circuitry which limits their ability to provide accurate bio-impedance measurements especially at higher measurement frequencies.

Solutions have been long sought but prior developments have not taught or suggested any complete solutions, and solutions to these problems have long eluded those skilled in the art. Thus, there remains a considerable need for devices and methods that can provide accurate high frequency bio-impedance measurements.

### BRIEF DESCRIPTION OF THE DRAWINGS

The bio-impedance measurement system is described and referenced herein as a “measurement system” and is illustrated in the figures of the accompanying drawings which are meant to be exemplary and not limiting, in which like reference numerals are intended to refer to like components, and in which:

FIG. 1 is a block diagram for a transmit block and a trans-impedance amplifier (TIA) block of the measurement system in a first embodiment.

FIG. 2 is a block diagram for a receive block of the first embodiment of FIG. 1, the second embodiment of FIG. 4, and the third embodiment of FIG. 5.

FIG. 3 is a timing diagram for the measurement system of FIG. 1.

FIG. 4 is a block diagram for a transmit block and a TIA block of the measurement system in a second embodiment.

FIG. 5 is a control flow for operating the measurement system.

FIG. 6 is a block diagram for a transmit block and a TIA block of the measurement system in a third embodiment.

FIG. 7 is a graphical plot of error for the measurement system of FIG. 5.

FIG. 8 is a timing diagram for the measurement system of FIG. 5.

FIG. 9 is a control flow for operating the measurement system of the third embodiment.

FIG. 10 is a control flow for manufacturing the measurement system.

### DETAILED DESCRIPTION

In the following description, reference is made to the accompanying drawings that form a part hereof, and in which are shown by way of illustration, embodiments in which the measurement system may be practiced. It is to be understood that other embodiments may be utilized and structural changes may be made without departing from the scope of the measurement system.

When features, aspects, or embodiments of the measurement system are described in terms of steps of a process, an operation, a control flow, or a flow chart, it is to be understood that the steps can be combined, performed in a different order, deleted, or include additional steps without departing from the measurement system as described herein.

The measurement system is described in sufficient detail to enable those skilled in the art to make and use the measurement system and provide numerous specific details to give a thorough understanding of the measurement system; however, it will be apparent that the measurement system may be practiced without these specific details.

In order to avoid obscuring the measurement system, some well-known system configurations and circuitry are not disclosed in detail; for example, those pertaining to measuring voltage, digitizing analog voltages, and performing digital calculations. When used in new, novel, and non-obvious ways, these new, novel, and non-obvious ways are described and set forth. Likewise, the drawings showing embodiments of the system are semi-diagrammatic and not to scale and, particularly, some of the dimensions are for the clarity of presentation and are shown greatly exaggerated in the drawing FIGS.

As used herein, the term “couple” as in “coupled” or “coupling” means a direct or indirect physical contact between coupled elements. As used herein, the term “connect” as in “connected” or “connecting” means a direct or indirect electrical connection between connected elements. The electrical connection can be made with electrically conductive traces, wires, or other electrically communicative means.

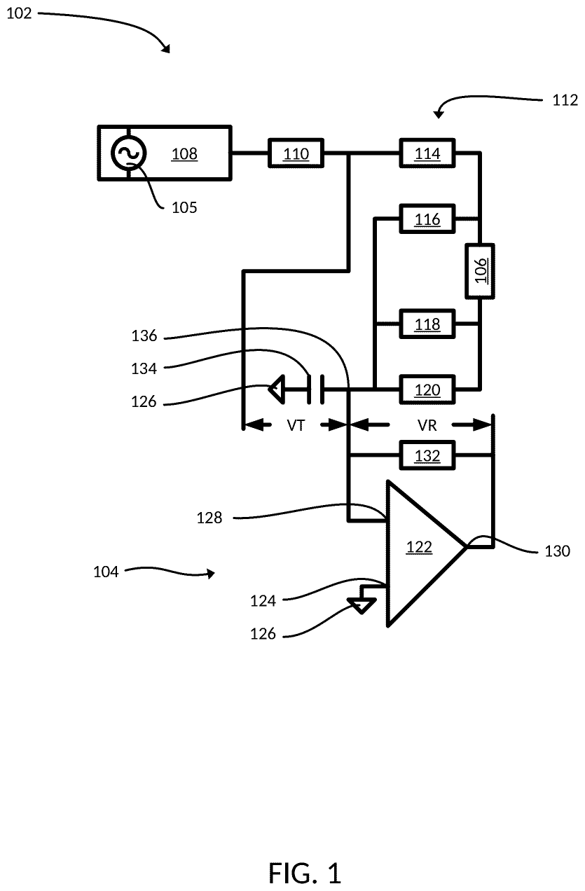

Referring now to FIG. 1, therein is shown a block diagram for a transmit block 102 and a trans-impedance amplifier (TIA) block 104 of the measurement system in a first embodiment.

The transmit block 102 can inject a sinusoidal transmit voltage signal, such as an AC voltage 105, into a body 106 of a measurement subject. The transmit block 102 can include a signal generator 108. The signal generator 108 can be a finite bandwidth sinusoidal voltage source coupled to a limiting resistor 110.

The limiting resistor 110 can be a safety resistor to limit maximum current to through the body 106 in the situation where a short occurs. This can ensure that the body 106 is not subject to unsafe levels of current.

The limiting resistor 110 can be determined based on the maximum output of the signal generator 108. Coupled to the limiting resistor 110 are electrodes 112 including a first electrode 114 having an impedance Z1, a second electrode 116 having an impedance Z2, a third electrode 118 having an impedance Z3, and a fourth electrode 120 having an impedance Z4. In the present configuration the first electrode 114 can be understood as a transmit electrode for injecting the AC voltage 105 while the second electrode 116, the third electrode 118, and the fourth electrode 120 can be understood as receive electrodes for receiving the AC voltage 105 after being transmitted through the body 106.

A bio-impedance (ZB) of the body 106 can be measured based on the electrodes 112 being in direct contact with the body 106. Illustratively, for example, the electrodes 112 could be implemented in a watch and the electrodes 112 could be in direct contact with the wrist of a measurement subject. While the bio-impedance ZB is described, this is intended to be a non-limiting illustration for descriptive purposes and the bio-impedance ZB of the body 106 could be any impedance external to the measurement system and the body 106 is contemplated to include objects other than human or animal bodies.

The impedances of the electrodes 112 can represent skin-electrode impedance and can change based on the condition of the electrodes 112 or the surface of the measurement subject in terms of dampness, roughness, and other contaminations. These changing impedances of the electrodes 112 can be a major source of inaccuracies when determining the bio-impedance ZB of the body 106.

The AC voltage 105 can be directed from the signal generator 108, through the limiting resistor 110, through the first electrode 114, and into the body 106. The AC voltage 105 can travel through the body 106 and back into the second electrode 116, the third electrode 118, and the fourth electrode 120.

The signal generator 108, the electrodes 112, and the limiting resistor 110 can be understood collectively as the transmit block 102. Connected to the transmit block 102 is the TIA block 104.

The TIA block 104 can include a TIA 122 including a first TIA input 124 connected to ground 126 a second TIA input 128 connected to the receive electrodes including the second electrode 116, the third electrode 118, and the fourth electrode 120. The TIA 122 is further shown having a TIA output 130.

The TIA 122 converts a current to voltage measured across a resistor RTIA 132, which can be connected between the second TIA input 128 and the TIA output 130. Ideally the TIA 122 will hold the second TIA input 128 to ground 126; however, in practice there is a parasitic capacitance 134 from circuitry within the transmit block 102 which results in a virtual ground 136. The difference between the ground 126 and the virtual ground 136 resulting from the parasitic capacitance 134 is a known source of measurement inaccuracy.

Transmit voltage measurements VT can be taken or acquired between the second TIA input 128 and the output of the limiting resistor 110. Receive voltage measurements VR can be taken or acquired between the TIA output 130 and the second TIA input 128, which is across the RTIA 132.

The receive voltage measurement VR and the transmit voltage measurement VT can be correlated to find the bio-impedance ZB of the body 106 and the electrodes 112 with Equation 1:

$$Z = RTIA * VT / VR \qquad Equation 1$$

Thus, the determination of impedance of the body 106 can be based on a ratio of the transmit voltage measurement VT and the receive voltage measurement VR as the impedances from the electrodes could be calculated through multiple measurements. It has been discovered that correlating only two measurements, including the transmit voltage measurement VT and the receive voltage measurement VR as a ratio also cancels errors resulting from measurement-to-measurement impedance changes of the signal generator 108 and the electrodes 112.

Yet furthermore, the correlated measurements between the transmit voltage measurement VT and the receive voltage measurement VR overcomes bandwidth limitations allowing the transmit block 102 to transmit at much higher frequencies than prior solutions. That is, prior solutions were limited in the upper range of frequencies that could be used due to capacitance on every electrode 112; however, the ratio of the transmit voltage measurement VT and the receive voltage measurement VR cancels these capacitances out and allows the transmit block 102 to be verifiably accurate at 3 MHz, which far exceeds all known solutions. It is expected that even higher frequencies are possible.

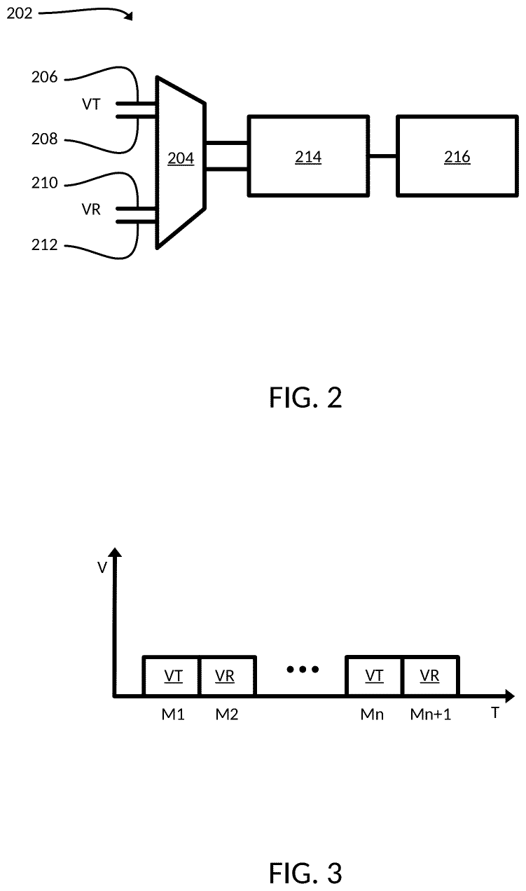

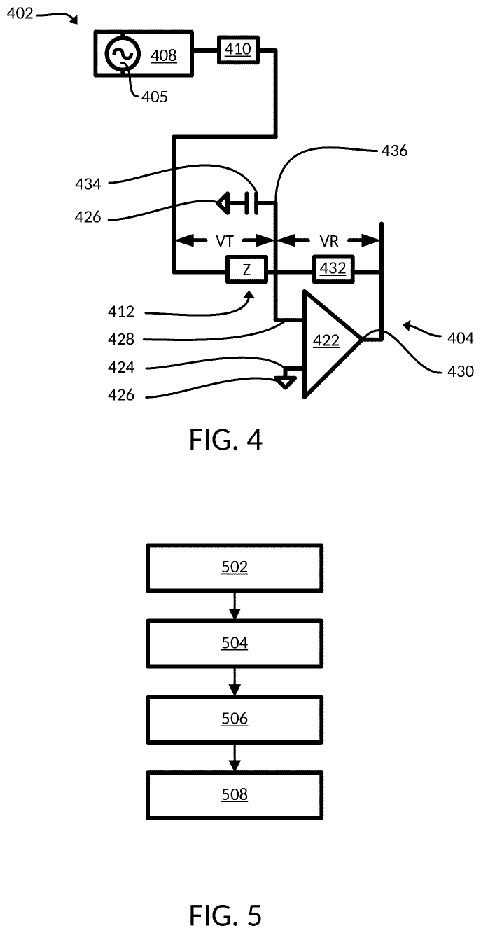

Referring now to FIG. 2, therein is shown a block diagram for a receive block 202 of the first embodiment of FIG. 1, the second embodiment of FIG. 4, and the third embodiment of FIG. 5. The receive block 202 can include a multiplexer 204 to switch between the transmit voltage measurement VT and the receive voltage measurement VR.

The multiplexer 204 should be understood as connected to between the second TIA input 128 of FIG. 1 and the output of the limiting resistor 110 of FIG. 1 for acquiring the transmit voltage measurement VT. The multiplexer 204 should further be understood as connected between the TIA output 130 of FIG. 1 and the second TIA input 128, which is across the RTIA 132 of FIG. 1 for acquiring the receive voltage measurement VR.

More particularly, the transmit voltage measurement VT can be acquired from a first connection 206 connected between the signal generator 108 and the first electrode 114 and from a second connection 208 connected to the second trans-impedance amplifier input 128. The receive voltage measurement VR can be acquired from a third connection 210 connected to the second trans-impedance amplifier input 128 and from a fourth connection 212 connected to the trans-impedance amplifier output 130.

The multiplexer 204 can provide either the transmit voltage measurement VT or the receive voltage measurement VR to voltage measurement circuitry 214. In some embodiments the voltage measurement circuitry 214 can include an instrumentation amplifier to sense a voltage difference between input terminals and output a voltage representative of that voltage difference. Some embodiments of the voltage measurement circuitry 214 can also include an analog to digital converter or ADC.

The output of the voltage measurement circuitry 214 can be a digital processing unit 216. The digital processing unit 216 can perform the calculations required to determine the bio-impedance ZB for the body 106 of FIG. 1. The digital processing unit 216 can include a processor for performing calculations of the transmit voltage measurement VT and the receive voltage measurement VR such as those of Equation 1 above or Equation 2 below.

Thus, the digital processing unit 216 performs calculations that are necessarily rooted in overcoming errors arising out of prior bio-impedance measurement technologies. That is, the digital processing unit 216 performing the calculations of Equation 1 or Equation 2 represent an improvement in providing more accurate bio-impedance measurements using the same transmit and receive channels, a solution which decreases the number of measurements and calibrations required and providing greater accuracy and enabling higher frequency measurements, and thus improving the bio-impedance measurement technologies.

It has been discovered that correlating the transmit voltage measurement VT and the receive voltage measurement VR using the same receive block 202 circuitry cancels receive circuit errors of the receive block 202 after the multiplexer 204. That is, the entire receive channel errors will cancel and there is no need for calibration.

It has been further discovered that any combination of circuitry used in the voltage measurement circuitry 214 will be cancelled. The correlated transmit voltage measurement VT and receive voltage measurement VR therefore provide cancellation from all circuitry induced errors of the receive block 202 and the transmit block 102 of FIG. 1, which greatly reduces complexity and provides an important and marketable improvement to impedance measurement technologies and products.

That is, by using the exact same measurement components, such as the same converter for transmit (ADCT) and receive (ADCR), any error in the reading from the ADC will cancel as shown by Equation 2:

$$Z = RTIA * VT / VR = RTIA * ADCT / ADCR \qquad Equation 2$$

Furthermore, by taking the transmit voltage measurement VT and the receive voltage measurement VR using the same receive circuitry, the bandwidth limitation of the receive circuitry is cancelled. This contributes to the ability of the disclosed measurement system to make precise and accurate readings at extremely high frequencies, which are unobtainable by prior solutions.

The important point in correlating the transmit voltage measurement VT and the receive voltage measurement VR is that a pair of VT and VR measurements are implemented using the exact same transmit block 102, TIA block 104 of FIG. 1 and the receive block 202.

Referring now to FIG. 3, therein is shown a timing diagram for the measurement system of FIG. 1. The timing diagram depicts voltage (V) along a vertical axis and time (T) along a horizontal axis.

The timing diagram further depicts a first measurement M1, which graphically depicts a measurement of the transmit voltage measurement VT of FIG. 1 made by the voltage measurement circuitry 214 and digitized by the digital processing unit 216, both of FIG. 2. A second measurement M2 is a graphical depiction of the receive voltage measurement VR of FIG. 1 made by the voltage measurement circuitry 214 and digitized by the digital processing unit 216.

The timing diagram illustrates the multiplexer 204 of FIG. 2 switching between the transmit voltage measurement VT and the receive voltage measurement VR for each measurement. That is, the first measurement M1 can be the transmit voltage measurements VT can be taken between the second TIA input 128 of FIG. 1 and the output of the limiting resistor 110 of FIG. 1. The second measurement M2 can be the receive voltage measurements VR can be taken between the TIA output 130 of FIG. 1 and the second TIA input 128, which is across the RTIA 132 of FIG. 1.

In some embodiments the time between the first measurement M1 and the second measurement M2 can be a matter of milliseconds. Multiple readings can be taken over time, but each set of readings includes a correlated measurement of the transmit voltage measurement VT and the receive voltage measurement VR. This is indicated by measurement Mn and Mn+1.

Referring now to FIG. 4, therein is shown a block diagram for a transmit block 402 and a TIA block 404 of the measurement system in a second embodiment. The receive block 202 of FIG. 2 can be used in conjunction with the transmit block 402 and the TIA block 404.

The transmit block 402 can inject a sinusoidal transmit voltage signal, such as an AC voltage 405, into a body of a measurement subject, for example that depicted by the body 106 of FIG. 1. The transmit block 402 can include a signal generator 408. The signal generator 408 can be a finite bandwidth sinusoidal voltage source coupled to a limiting resistor 410.

The limiting resistor 410 can be a safety resistor to limit maximum current to through the body in the situation where a short occurs. This can ensure that the body is not subject to unsafe levels of current.

The limiting resistor 410 can be determined based on the maximum output of the signal generator 408. Coupled to the limiting resistor 410 are electrodes 412. The electrodes 412 are graphically depicted as a single block to indicate any electrode configuration with the impedance Z together with the bio-impedance ZB based on the electrodes 412 being in contact with a body.

For example, the electrodes 412 can indicate two electrodes with individual impedances together with the bio-impedance ZB of a body. It has been discovered that utilizing the same transmit block 402, the same TIA block 404, and the same receive block 202 results in the cancellation of any non-idealities arising from each of these blocks.

While the electrodes 412 are described as a two electrode configuration, any configuration or number of electrodes may be used or substituted. Illustratively, two three, five, six, or more electrodes could be used as the electrodes 412, although the two electrode configuration described is known to be less accurate than the four electrode configuration depicted in FIG. 1.

The bio-impedance ZB of a body can be measured based on the electrodes 412 being in direct contact with a body. Illustratively, for example, the electrodes 412 could be implemented in a watch and the electrodes 412 could be in direct contact with the wrist of a measurement subject.

The impedances of the electrodes 412 can represent skin-electrode impedance and can change based on the condition of the electrodes 412 or the surface of the measurement subject in terms of dampness, roughness, contaminations, and completeness of electrode-skin contact. These changing impedances of the electrodes 412 can be a major source of inaccuracies when determining the bio-impedance ZB of the body.

The AC voltage 405 can be directed from the signal generator 408, through the limiting resistor 410 and through the body based on the electrodes 412 being in direct contact with the body. The signal generator 408, the electrodes 412, and the limiting resistor 410 can be understood collectively as the transmit block 402. Connected to the transmit block 402 is the TIA block 404.

The TIA block 404 can include a TIA 422 including a first TIA input 424 connected to ground 426 a second TIA input 428 connected to receive electrodes. The TIA 422 is further shown having a TIA output 430.

The TIA 422 converts a current to voltage measured across a resistor RTIA 432, which can be connected between the second TIA input 428 and the TIA output 430. Ideally the TIA 422 will hold the second TIA input 428 to ground 426; however, in practice there is a parasitic capacitance 434 from circuitry within the transmit block 402 which results in a virtual ground 436. The difference between the ground 426 and the virtual ground 436 resulting from the parasitic capacitance 434 is a known source of measurement inaccuracy.

Transmit voltage measurements VT can be taken between the second TIA input 428 and the output of the limiting resistor 410. Receive voltage measurements VR can be taken between the TIA output 430 and the second TIA input 428, which is across the RTIA 432 and is connected to receive electrodes configured to receive AC voltage 405 from the body. The receive voltage measurement VR and the transmit voltage measurement VT can be correlated to find the bio-impedance ZB of the body with Equation 1 even while using any configuration of electrodes represented by electrodes 412.

It has been discovered that correlating only two measurements, including the transmit voltage measurement VT and the receive voltage measurement VR, can be used as a ratio which cancels errors resulting from measurement-to-measurement impedance changes of the signal generator 408 and the electrodes 412. Furthermore, prior solutions required calibration routines, which are not needed when taking the ratio of the transmit voltage measurement VT and the receive voltage measurement VR.

Yet furthermore, the correlated measurements between the transmit voltage measurement VT and the receive voltage measurement VR overcomes bandwidth limitations allowing the transmit block 402 to transmit at much higher frequencies than prior solutions. That is, prior solutions were limited in the upper range of frequencies that could be used due to capacitance on every electrode 412; however, the ratio of the transmit voltage measurement VT and the receive voltage measurement VR cancels these capacitances out and allows the transmit block 402 to be verifiably accurate at frequencies that far exceeds all known solutions.

Referring now to FIG. 5, therein is shown a control flow for operating the measurement system. The control flow can include applying an AC voltage through a body connected to electrodes in a block 502; acquiring a transmit voltage measurement measured across the electrodes in a block 504; acquire a receive voltage measurement across a resistor connected between a trans-impedance amplifier input and a trans-impedance amplifier output in a block 506; and calculate an impedance based on the electrodes being in direct contact with a body, the impedance based on a ratio of the transmit voltage measurement and the receive voltage measurement in a block 508, and wherein calculating the impedance includes calculating the impedance based on Equation 1.

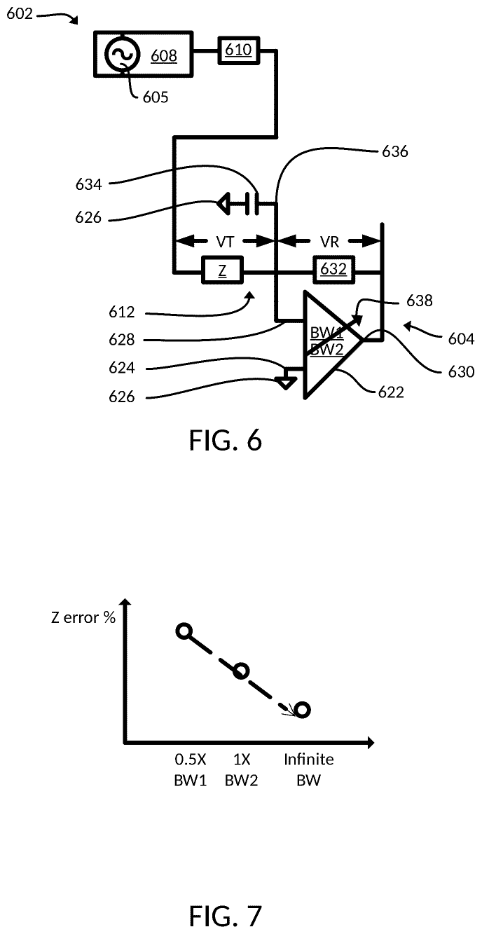

Referring now to FIG. 6, therein is shown a block diagram for a transmit block 602 and a TIA block 604 of the measurement system in a third embodiment. The receive block 202 of FIG. 2 can be used in conjunction with the transmit block 602 and the TIA block 604.

The transmit block 602 can inject a sinusoidal transmit voltage signal, such as an AC voltage 605, into a body of a measurement subject, for example that depicted by the body 106 of FIG. 1. The transmit block 602 can include a signal generator 608. The signal generator 608 can be a finite bandwidth sinusoidal voltage source coupled to a limiting resistor 610.

The limiting resistor 610 can be a safety resistor to limit maximum current to through the body in the situation where a short occurs. This can ensure that the body is not subject to unsafe levels of current.

The limiting resistor 610 can be determined based on the maximum output of the signal generator 608. Coupled to the limiting resistor 610 are electrodes 612. The electrodes 612 are graphically depicted as a single block to indicate any electrode configuration with the impedance Z together with the bio-impedance ZB based on the electrodes 612 being in contact with a body.

For example, the electrodes 612 can indicate two electrodes with individual impedances together with the bio-impedance ZB of a body. It has been discovered that utilizing the same transmit block 602, the same TIA block 604, and the same receive block 202 results in the cancellation of the impedance of the electrodes 612 leaving the bio-impedance ZB measurable as a result.

While the electrodes 612 are described as a two electrode configuration, any configuration or number of electrodes may be used or substituted. Illustratively, two three, four, five, six, or more electrodes could be used as the electrodes 612. In the present configuration electrodes that inject or transmit the AC voltage 605 into the body can be understood as transmit electrodes while the electrodes that receive the AC voltage 605 from the body can be understood as receive electrodes for receiving the AC voltage 605 after being transmitted through the body.

The bio-impedance ZB of a body can be measured based on the electrodes 612 being in direct contact with a body. Illustratively, for example, the electrodes 612 could be implemented in a watch and the electrodes 612 could be in direct contact with the wrist of a measurement subject.

The impedances of the electrodes 612 can represent skin-electrode impedance and can change based on the condition of the electrodes 612 or the surface of the measurement subject in terms of dampness, contaminations, roughness, and completeness of electrode-skin contact. These changing impedances of the electrodes 612 can be a major source of inaccuracies when determining the bio-impedance ZB of the body.

The AC voltage 605 can be directed from the signal generator 608, through the limiting resistor 610 and through the body based on the electrodes 612 being in direct contact with the body. The signal generator 608, the electrodes 612, and the limiting resistor 610 can be understood collectively as the transmit block 602. Connected to the transmit block 602 is the TIA block 604.

The TIA block 604 can include a TIA 622 including a first TIA input 624 connected to ground 626 a second TIA input 628 connected to receive electrodes. The TIA 622 is further shown having a TIA output 630.

The TIA 622 converts a current to voltage measured across a resistor RTIA 632, which can be connected between the second TIA input 628 and the TIA output 630. Ideally the TIA 622 will hold the second TIA input 628 to ground 626; however, in practice there is a parasitic capacitance 634 from circuitry within the transmit block 602 which results in a virtual ground 636. The difference between the ground 626 and the virtual ground 636 resulting from the parasitic capacitance 634 is a known source of measurement inaccuracy.

Transmit voltage measurements VT can be taken between the second TIA input 628 and the output of the limiting resistor 610. Receive voltage measurements VR can be taken between the TIA output 630 and the second TIA input 628, which is across the RTIA 632 and is connected to receive electrodes configured to receive AC voltage 605 from the body.

The receive voltage measurement VR and the transmit voltage measurement VT can be correlated to find the bio-impedance ZB of the body with Equation 1 even while using any configuration of electrodes represented by electrodes 612. The TIA 622 is further depicted with a bandwidth selector 638 for acquiring the transmit voltage measurement VT the receive voltage measurement VR for two different bandwidths.

Illustratively for example, the bandwidth selector 638 can representatively depict an internal selectable compensation capacitor within the TIA 622 and which can reduce the bandwidth of the TIA 622. The compensation capacitor can be changed by a known ratio. Alternatively, the bandwidth selector 638 can representatively depict input transistors within the TIA 622, which can be adjusted to change the bias current and thereby change the bandwidth of the TIA 622.

It has been discovered that when implemented as a resistor, the transmit voltage measurement VT and the receive voltage measurement VR could be correlated using the same signal path. That is, the receive voltage measurement VR and transmit voltage measurement VT could be acquired through one bandwidth signal path and a second receive voltage measurement VR and a second receive voltage measurement VR could be acquired through the second bandwidth signal path.

Thus, two bandwidths for transmit voltage measurement VT and receive voltage measurement VR could be obtained using the exact same circuitry for each individual bandwidth measurement and the transmit voltage measurement VT and the receive voltage measurement VR for each bandwidth will correlate as a ratio, which cancels errors resulting from measurement-to-measurement impedance changes of the signal generator 608 and the electrodes 612.

In addition to these improvements, it has been discovered that multiple bandwidth measurements of the transmit voltage measurement VT and the receive voltage measurement VR can greatly reduce the error generated by the parasitic capacitance 634, which represents the number one source of error as the frequency of the AC voltage 605 increases and is the only remaining source of off chip error remaining after the transmit voltage measurement VT and the receive voltage measurement VR are correlated, as described above.

Therefore, it has been discovered that taking correlated measurements of the transmit voltage measurement VT and the receive voltage measurement VR at different bandwidths represents an important improvement enabling the measurement system to operate at even greater frequencies and fixes the error seen between the virtual ground 636 and ground 626. Although the bandwidth selector 638 is described as reducing the bandwidth of the AC voltage by half, other bandwidth ratios are contemplated, and this bandwidth ratio serves only as a non-limiting illustration.

Referring now to FIG. 7, therein is shown a graphical plot of error for the measurement system of FIG. 5. The graphical plot depicts impedance error of the electrodes 612 of FIG. 6 as a percentage along a vertical axis and bandwidth along a horizontal axis.

An open loop math method can be used to extrapolate the impedance based on known bandwidth ratios. One illustrative example is described below in Equation 3.

The graphical plot illustratively depicts a half band width BW1 and a full bandwidth BW2 having a ratio of 2X. As will be appreciated, since the open loop math method is ratio based, the open loop math method is Beta independent meaning the feedback factor of the loop is the same for BW1 and BW2.

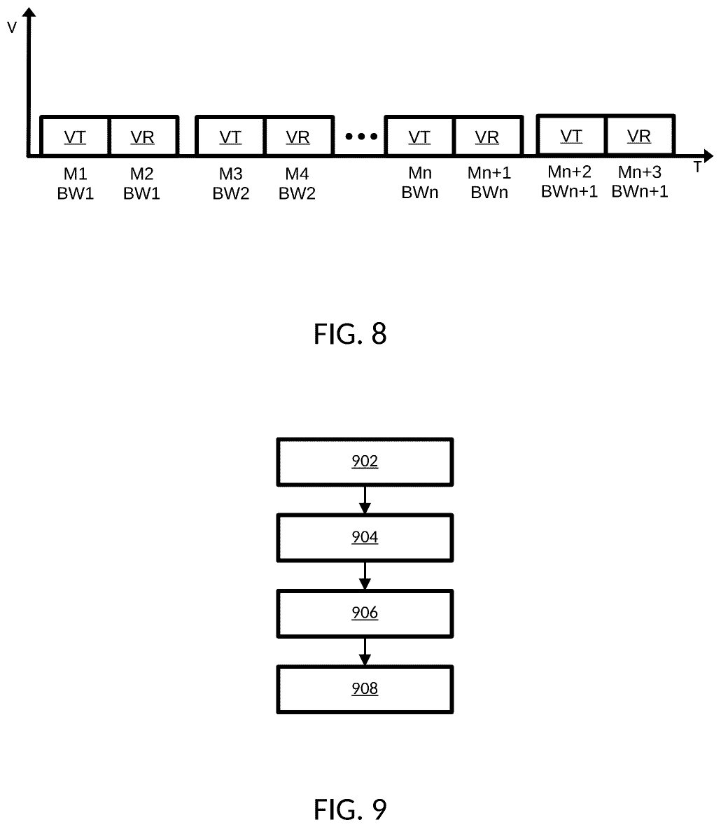

Referring now to FIG. 8, therein is shown a timing diagram for the measurement system of FIG. 5. The timing diagram depicts voltage (V) along a vertical axis and time (T) along a horizontal axis.

The timing diagram further depicts a first measurement M1 at a first bandwidth BW1, which graphically depicts a measurement of the transmit voltage measurement VT of FIG. 1 made by the voltage measurement circuitry 214 and digitized by the digital processing unit 216, both of FIG. 2. A second measurement M2 at the first bandwidth BW1 is a graphical depiction of the receive voltage measurement VR of FIG. 1 made by the voltage measurement circuitry 214 and digitized by the digital processing unit 216.

The timing diagram illustrates the multiplexer 204 of FIG. 2 switching between the transmit voltage measurement VT and the receive voltage measurement VR for M1 and M2 at the first bandwidth BW1. The timing diagram yet further depicts a third measurement M3 at a second bandwidth BW2, which graphically depicts a measurement of the transmit voltage measurement VT made by the voltage measurement circuitry 214 and digitized by the digital processing unit 216. A fourth measurement M4 at the second bandwidth BW2 is a graphical depiction of the receive voltage measurement VR made by the voltage measurement circuitry 214 and digitized by the digital processing unit 216.

The timing diagram illustrates the multiplexer 204 switching between the transmit voltage measurement VT and the receive voltage measurement VR for M3 and M4 at the second bandwidth BW2.

In some embodiments the time between the first measurement M1 and the second measurement M2 can be a matter of milliseconds. The impedance from the first bandwidth BW1 and the second bandwidth BW2 can be correlated by ratio to greatly reduce error from parasitic capacitance 634 of FIG. 6.

Multiple groups of readings can be taken over time, but each set of readings includes a correlated measurement of the transmit voltage measurement VT and the receive voltage measurement VR and these measurements are further correlated for similar measurements at differing bandwidths. This is indicated by measurement Mn at BWn, Mn+1 at BWn, Mn+2 at BWn+1, and Mn+3 at BWn+1.

The error caused by finite bandwidth is, to the first order, proportional to the TIA bandwidth. Because the ratio of BW1 and BW2 is known, the error caused by finite bandwidth can be eliminated using linear interpolation. For illustrative purposes, using only two bandwidths where BW1 is twice the BW2, as shown in FIG. 7 above, the bio-impedance ZB can be calculated using Equation 3:

$$ZB = 2 * ZB 1 - ZB 2 \qquad Equation 3$$

where ZB1 is an estimated bio-impedance ZB at BW1 or full bandwidth and ZB2 is an estimated bio-impedance ZB at BW2 or half bandwidth.

It has been discovered that the bio-impedance ZB measurement caused by finite bandwidth of the TIA has a linear relation to the TIA bandwidth in the 1^{st} order described by Equation 4:

$$r = ( Z ( lowBW ) - Z ( ideal ) ) / ( Z ( highBW ) - Z ( ideal ) ) \qquad Equation 4$$

where r is the bandwidth ratio. Therefore, when r=2, the corrected bio-impedance ZB is described by Equation 5:

$$ZB = ( rZ ( fullBW ) - Z ( halfBW ) ) / ( r - 1 ) \qquad Equation 5$$

Equation 5 then reduces to Equation 3. It will be appreciated that Equation 4 can be used to calculate a corrected bio-impedance ZB with other ratios of the TIA bandwidth.

Referring now to FIG. 9, therein is shown a control flow for operating the measurement system of the third embodiment. The control flow can include acquiring the transmit voltage measurement VT and the receive voltage measurement VR at a first bandwidth, which can be the highest bandwidth of the measurement system, for the AC voltage in a block 902; changing the bandwidth of the TIA to a second bandwidth in a block 904; acquiring the transmit voltage measurement VT and the receive voltage measurement VR at the second bandwidth in a block 906; calculate the bio-impedance ZB in a block 908.

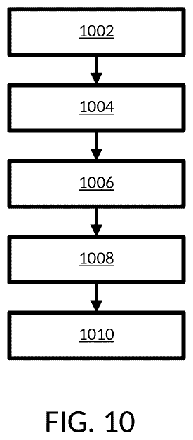

Referring now to FIG. 10, therein is shown a control flow for manufacturing the measurement system. The control flow can include providing a signal generator configured to provide a transmit voltage in a block 1002; connecting a transmit electrode to the signal generator in a block 1004; providing a receive electrode in a block 1006; connecting a trans-impedance amplifier input of a trans-impedance amplifier to the receive electrode, the trans-impedance amplifier having a trans-impedance amplifier output in a block 1008; providing a voltage measurement circuitry configured to: acquire a transmit voltage measurement from a first connection connected between the signal generator and the transmit electrode and from a second connection connected to the trans-impedance amplifier input, and acquire a receive voltage measurement from a third connection connected to the trans-impedance amplifier input and from a fourth connection connected to the trans-impedance amplifier output in a block 1010; and connecting a digital processing unit to the voltage measurement circuitry, the digital processing unit configured to determine an impedance based on the transmit electrode and the receive electrode being in direct contact with a body, the impedance based on a ratio of the transmit voltage measurement and the receive voltage measurement in a block 1012.

Thus, it has been discovered that the measurement system furnishes important and heretofore unknown and unavailable solutions, capabilities, and functional aspects. The resulting configurations are straightforward, cost-effective, uncomplicated, highly versatile, accurate, sensitive, and effective, and can be implemented by adapting known components for ready, efficient, and economical manufacturing, application, and utilization.

While the measurement system has been described in conjunction with a specific best mode, it is to be understood that many alternatives, modifications, and variations will be apparent to those skilled in the art in light of the preceding description. Accordingly, it is intended to embrace all such alternatives, modifications, and variations, which fall within the scope of the included claims. All matters set forth herein or shown in the accompanying drawings are to be interpreted in an illustrative and non-limiting sense.

What is claimed is:

1. A measurement system comprising:
a signal generator configured to provide a transmit voltage;
a transmit electrode connected to the signal generator;
a trans-impedance amplifier having a trans-impedance amplifier input and a trans-impedance amplifier output;
a receive electrode connected to the trans-impedance amplifier input;
voltage measurement circuitry configured to:
acquire a transmit voltage measurement from a first connection connected between the signal generator and the transmit electrode and from a second connection connected to the trans-impedance amplifier input, and
acquire a receive voltage measurement from a third connection connected to the trans-impedance amplifier input and from a fourth connection connected to the trans-impedance amplifier output; and
a digital processing unit configured to determine an impedance based on the transmit electrode and the receive electrode being in direct contact with a body, the impedance based on a ratio of the transmit voltage measurement and the receive voltage measurement.

2. The system of claim 1 further comprising:
a trans-impedance amplifier bandwidth selector; and
wherein:
the voltage measurement circuitry is configured to acquire the transmit voltage measurement and the receive voltage measurement for different bandwidths of the trans-impedance amplifier.

3. The system of claim 1 further comprising: a multiplexer connected to the voltage measurement circuitry configured to switch to the first connection and the second connection for acquiring the transmit voltage measurement and switch to the third connection and the fourth connection for acquiring the receive voltage measurement.

4. The system of claim 1 wherein: the signal generator is a finite bandwidth sinusoidal voltage source.

5. The system of claim 1 further comprising: a limiting resistor between the signal generator and the transmit electrode, and wherein the first connection is connected between the limiting resistor and the transmit electrode.

6. The system of claim 1 wherein: the receive electrode is one of three receive electrodes.

7. The system of claim 1 wherein: the digital processing unit is configured to determine a bio-impedance of the body.

8. The system of claim 1 wherein: the voltage measurement circuitry includes an analog to digital converter configured to acquire both the transmit voltage measurement and the receive voltage measurement.

9. The system of claim 1 further comprising: a trans-impedance amplifier resistor connected to the trans-impedance amplifier input and the trans-impedance amplifier output.

10. The system of claim 9 wherein: the digital processing unit is configured to determine the impedance based on Equation 1.

11. A method of manufacturing a measurement system comprising:
providing a signal generator configured to provide a transmit voltage;
connecting a transmit electrode to the signal generator;
providing a receive electrode;
connecting a trans-impedance amplifier input of a trans-impedance amplifier to the receive electrode, the trans-impedance amplifier having a trans-impedance amplifier output;
providing voltage measurement circuitry configured to:
acquire a transmit voltage measurement from a first connection connected between the signal generator and the transmit electrode and from a second connection connected to the trans-impedance amplifier input, and
acquire a receive voltage measurement from a third connection connected to the trans-impedance amplifier input and from a fourth connection connected to the trans-impedance amplifier output; and
connecting a digital processing unit to the voltage measurement circuitry, the digital processing unit configured to determine an impedance based on the transmit electrode and the receive electrode being in direct contact with a body, the impedance based on a ratio of the transmit voltage measurement and the receive voltage measurement.

12. The method of claim 11 wherein: connecting the trans-impedance amplifier includes connecting the trans-impedance amplifier having a trans-impedance amplifier bandwidth selector; and
providing the voltage measurement circuitry includes providing the voltage measurement circuitry configured to acquire the transmit voltage measurement and the receive voltage measurement for different bandwidths of the trans-impedance amplifier.

13. The method of claim 11 further comprising: connecting a multiplexer to the voltage measurement circuitry configured to switch to the first connection and the second connection for acquiring the transmit voltage measurement and switch to the third connection and the fourth connection for acquiring the receive voltage measurement.

14. The method of claim 11 wherein: providing the signal generator includes providing a finite bandwidth sinusoidal voltage source.

15. The method of claim 11 further comprising:
connecting a limiting resistor between the signal generator and the transmit electrode; and
wherein:
providing the voltage measurement circuitry configured to acquire the transmit voltage measurement from the first connection includes providing the voltage measurement circuitry configured to acquire the transmit voltage measurement from the first connection connected between the limiting resistor and the transmit electrode.

16. The method of claim 11 wherein: connecting the electrodes includes connecting the receive electrode as one of three receive electrodes.

17. The method of claim 11 wherein: connecting the digital processing unit includes connecting the digital processing unit configured to determine a bio-impedance of the body.

18. The method of claim 11 wherein: providing the voltage measurement circuitry includes providing the voltage measurement circuitry having an analog to digital converter configured to acquire both the transmit voltage measurement and the receive voltage measurement.

19. The method of claim 11 further comprising: connecting a trans-impedance amplifier resistor to the trans-impedance amplifier input and the trans-impedance amplifier output.

20. The method of claim 19 wherein: connecting the digital processing unit includes connecting the digital processing unit configured to determine the impedance based on Equation 1.

21. A method of operating a measurement system comprising:
applying an AC voltage through a body connected to electrodes;
acquiring a transmit voltage measurement measured across the electrodes;
acquiring a receive voltage measurement across a resistor connected between a trans-impedance amplifier input and a trans-impedance amplifier output of a trans-impedance amplifier; and
calculating an impedance based on the electrodes being in direct contact with a body, the impedance based on a ratio of the transmit voltage measurement and the receive voltage measurement.

22. The method of claim 21 wherein: calculating the impedance includes calculating the impedance based on Equation 1.

23. The method of claim 21 wherein:
acquiring the transmit voltage measurement includes acquiring a first measurement at a first bandwidth of the trans-impedance amplifier;
acquiring the receive voltage measurement includes acquiring a second measurement at the first bandwidth of the trans-impedance amplifier; and
further comprising:
changing the trans-impedance amplifier to operate with a second bandwidth;
acquiring a third measurement, the third measurement being the transmit voltage measurement at the second bandwidth of the trans-impedance amplifier; and
acquiring a fourth measurement, the fourth measurement being the receive voltage measurement at the second bandwidth of the trans-impedance amplifier.
